(19) United States
(12) Patent Application Publication
(10) Pub. No.: US 2024/0392254 A1
(43) Pub. Date: Nov. 28, 2024

(54) CANCER MICROENVIRONMENT

(71) Applicant: CARCINOTECH LTD, Midlothian (GB)

(72) Inventor: Ishani MALHOTRA, Edinburgh (GB)

(21) Appl. No.: 18/704,559

(22) PCT Filed: Oct. 25, 2022
(86) PCT No.: PCT/GB2022/052717

§ 371 Date: Apr. 25, 2024

(30) Foreign Application Priority Data
Oct. 25, 2021 (GB) 2115337.4

(51) Int. Cl. C12N 5/071; C12N 5/0786; C12N 5/079; C12N 5/095; G01N 33/50

(52) U.S. Cl. CPC C12N 5/0697; C12N 5/0622; C12N 5/0645; C12N 5/0695; G01N 33/5082; C12N 2513/00; C12N 2533/74; C12N 2533/78; G01N 2800/7028

(57) ABSTRACT

The present disclosure relates to constructs, and particularly bioprinted constructs, that are able to mimic an in vivo environment, such a cancer microenvironment. The disclosure also extends to methods and kits for making such constructs. The constructs may find particular application as models for drug development, drug screening and/or clinical evaluation of a drug product.

### FIELD

The present disclosure relates to models or constructs that are able to mimic an in vivo environment, such a cancer microenvironment. The disclosure also extends to methods of making such models or constructs, kits for making the described models or constructs and uses of the models or constructs.

### BACKGROUND

Cancer stem cells (CSCs) are tumour cells which have the property of stem cells and are believed to play an important role in tumour initiation and progression. Cancer stem cells reside in specialized microenvironments called niches, which can reside within tumour microenvironments. CSC niches regulate and modulate the properties of CSCs, help maintain the CSC phenotype and shield them from the body's immune system.

Bioengineered constructs or artificial models are of increasing importance in the development of new therapies or drugs and offer the opportunity to reduce the reliance on animal models. However, it is not trivial to provide a construct or model that accurately mimics an in vivo environment. This is particularly true for a CSC niche or tumour microenvironment, which are highly complex.

Many cancer models involve 2D and/or 3D cell cultures in conventional plastic dishes or even on a gel matrix. More recent studies may also involve the use of scaffolds to study 3D structures of cancers and their interactions with other cells. A recent publication (WO 2020/254660 A1) provides a specialised microfluidic device for mimicking a cancer microenvironment. In addition, Hermida et al (“Three dimensional in vitro models of cancer: Bioprinting multilineage glioblastoma models”; Advances in Biological Regulation 75 (2020), 100658) and Tang et al (“Three dimensional bioprinted glioblastoma microenvironments model cellular dependencies and immune interactions”; Cell Research (2020) 30: 833-853) describe bioprinted models of glioblastoma using multiple cell types.

However, there remains a need to provide artificial constructs or models that can mimic a real-life cancer microenvironment, that can mimic a cancer heterogeneity, and/or that can be made quickly and/or cost effectively.

### SUMMARY

According to an aspect of the disclosure, there is provided a construct for mimicking an in vivo environment, such as an in vivo cancer microenvironment. The construct may be a tumour construct that is able to replicate an in vivo cancer microenvironment and so provide a representative model of tumour growth and development. Such constructs may find particular application in drug assays (e.g. during drug testing and development) and/or preclinical studies. The disclosure can exploit many different types of cell to provide constructs that are able to mimic many different types of cancer microenvironment.

The construct may comprise at least one of each of the following:
(a) a cancer cell;
(b) a cancer stem cell; and
(c) a cancer associated fibroblast cell.

It should be noted that the terms “comprise”, “comprising” and/or “comprises” is/are used to denote that aspects and embodiments of this invention “comprise” a particular feature or features. It should be understood that this/these terms may also encompass aspects and/or embodiments which “consist essentially of” or “consist of” the relevant feature or features. Accordingly, in some examples, the construct may consist (or consist essentially of) at least one of each of the cells (a) to (c) listed above.

The constructs of the disclosure are in vitro microenvironments or artificial models or constructs that have the capacity to mimic an in vivo microenvironment.

The present inventors have unexpectedly identified that relatively simple constructs based on at least the three core cell types (a) to (c) noted above can provide realistic models of a cancer microenvironment. In particular, the constructs as described herein can be used to provide cancer models with an increased complexity that more closely reflect a real-life tumour microenvironment. In addition, in comparison to simpler models (e.g. those based on two cell types), the constructs disclosed herein may be prepared far more rapidly. In particular, constructs of the present disclosure can be ready for use in drug assays between about 7 to 21 days, e.g. within around 14 days.

In some examples, in addition to the cell types (a) to (c) noted above, the construct may further comprise one or more of the following:
(e) a supportive cell;
(f) a specialized cell type to provide a specific microenvironment;
(g) an immune cell; and/or
(h) an endothelial cell.

In such examples, the construct may consist (or consist essentially) of the cells (a) to (c), together with one or more of the following:
(e) a supportive cell;
(f) a specialized cell type to provide a specific microenvironment;
(g) an immune cell; and/or
(h) an endothelial cell.

In some examples, the construct may further comprise an extracellular matrix together with the core cell types (a) to (c) (and optionally one or more of cells (e) to (h)). By way of example only, the construct may consist (or consist essentially of) cells (a) to (c) (with optionally one or more of cells (e) to (h)) together with (d) an extracellular matrix.

In the described constructs, the cancer cell (e.g. component (a)) may be selected from any known cancer cells and/or may be a patient-derived, established or primary cell type. Representative examples include, but are not limited to, brain cancer cells, lung cancer cells, breast cancer cells, prostate cancer cells, colorectal cancer cells, ovarian cancer cells, pancreatic cancer cells, skin cancer cells, bone cancer cells and the like. For example, the cancer cell may be selected from the group consisting of brain tumour cells, breast cancer cells and lung cancer cells. The cancer cell may be a glioblastoma cell such as U-87 (sometimes referred to as U87-MG, Uppsala 87 Malignant Glioma). The cancer cell may be a breast cancer cell (e.g. a triple negative breast cancer such as MDA-MB-231 cancer cells). MDA-MB-231 is an epithelial human breast cancer cell line. MDA-MB-231 is a highly aggressive, invasive and poorly differentiated triple-negative breast cancer (TNBC) cell line as it lacks oestrogen receptor (ER) and progesterone receptor (PR) expression, as well as HER2 (human epidermal growth factor receptor 2) amplification.

The cancer stem cell (e.g. component (b)) may be selected from any known cancer stem cells and/or may be a patient-derived or a primary cell type. Representative examples include, but are not limited to, brain cancer stem cells, lung cancer stem cells, breast cancer stem cells, prostate cancer stem cells, colorectal cancer stem cells, ovarian cancer stem cells, pancreatic cancer stem cells, skin cancer stem cells, bone cancer stem cells and the like. For example, the cancer stem cell may be selected from the group consisting of brain tumour stem cells, breast cancer stem cells and lung cancer stem cells. In some examples, the cancer stem cell may be a glioblastoma cancer stem cell (GBM CSC). In other examples, the cancer stem cell may be a breast cancer stem cell (BCSC).

The cancer associated fibroblast cell (e.g. component (c)) may be selected from any known cancer associated fibroblast cell and/or may be a patient-derived or a primary cell type. Representative examples include, but are not limited to, brain cancer associated fibroblast cells, lung cancer associated fibroblast cells, breast cancer associated fibroblast cells, prostate cancer associated fibroblast cells, colorectal cancer associated fibroblast cells, ovarian cancer associated fibroblast cells, pancreatic cancer associated fibroblast cells, skin cancer associated fibroblast cells, bone cancer associated fibroblast cells and the like. For example, the cancer associated fibroblast cell may be selected from the group consisting of brain tumour associated fibroblast cells, breast cancer associated fibroblast cells and lung cancer associated fibroblast cells. In some examples, the cancer associated fibroblast cell may be a glioblastoma cancer associated fibroblast cell (GBM CAF). In some examples, the cancer associated fibroblast cell may be a breast cancer associated fibroblast cell (BC CAF).

As will be appreciated by the skilled person, the specific nature of cells (a) to (c) and optionally (e) to (h) may be selected in accordance with the type of cancer microenvironment that the construct is intended to reflect. By way of example only, where the construct is to mimic the in vivo microenvironment of a brain tumour, component (a) may be a glioblastoma, component (b) may be a glioblastoma cancer stem cell and component (c) may be a glioblastoma cancer associated fibroblast cell. By way of a further example, where the construct is to mimic the in vivo microenvironment of a breast cancer (such as a triple negative breast cancer), component (a) may be a breast cancer cell, component (b) may be a breast cancer stem cell, and component (c) may be a breast cancer associated fibroblast cell.

A supportive cell (e.g. component (e)) may be selected from any cell type that functions to provide structural support to the construct. By way of example only, the supportive cell may be selected from epithelial cells and astrocytes (e.g. Immortalized Human Astrocytes (IM-HA) obtained from Innoprot IHACLON4).

A specialized cell type (e.g. component (f)) to provide a specific microenvironment may be selected in accordance with the type of cancer microenvironment that the structure is intended to mimic or reflect. By way of example only, where the construct is to mimic the in vivo microenvironment of a brain tumour, component (f) may be a microglial cell such as HMC3).

Other examples may include adipocyte cells. A representative example of adipocyte cells is human adipocyte cells, such as the cell line hMAds.

The inclusion of immune cells (e.g. component (g)) in the construct may be of particular utility where the construct is intended for use in for immuno-oncological studies. In such examples, the immune cells may be selected from lymphocytes, macrophages and the like. By way of example only, where the construct is intended to mimic the in vivo microenvironment of a brain tumour, the immune cells may be selected from tumour infiltrating lymphocytes (TILs) and tumour associated macrophages (TAMs), or combinations thereof. The immune cells may be patient-derived or a primary cell type. In some examples, the immune cells may be patient-derived.

In some examples, the construct may further comprise (h) endothelial cells. The inclusion of endothelial cells may be useful in promoting vascularization within the construct.

By way of further example only, where the construct is to mimic the in vivo microenvironment of a brain tumour, the construct may comprise, consist essentially or consist of five core cell types: a brain tumour cell, a brain tumour stem cell, a brain tumour associated fibroblast cell, astrocyte and microglia. In one example, component (a) is a glioblastoma, component (b) is a glioblastoma cancer stem cell, component (c) is a glioblastoma cancer associated fibroblast cell, component (e) is an astrocyte and component (f) is a microglial cell.

By way of additional example, where the construct is to mimic the in vivo microenvironment of a breast cancer, the construct may comprise, consist essentially or consist of four core cell types: a breast cancer cell, a breast cancer stem cell, a breast cancer associated fibroblast cell and an adipocyte cell (such as a human adipocyte cell).

As used herein, patient-derived cells may refer to cells that have been obtained and/or sourced from a patient (e.g. a patient suffering from one of the cancers noted herein). The use of such cells in the described constructs may facilitate screening of test agents in models that more closely reflect the tumour characteristics of a particular patient and/or provide a more accurate prediction of a clinical response in a particular patient.

As will be understood from the above, the construct may comprise (or be formed from) a population of cells of types (a), (b) and (c), and also of one or more of types (e) to (h) where present. Within the population of cells, each cell type may be included in a proportion that facilitates and/or promotes the growth and/or development of a construct that mimics the in vivo cancer microenvironment.

In some examples, cancer cells may comprise between about 20% and 90%, between about 30% and 80%, between about 40% and 70%, between about 45% and 65%, or between about 50% and 60% of the cell population. In some examples, cancer cells may comprise between about 60% to 80%, or between about 65% and 75% of the cell population (e.g. about 70% of the cell population).

In some examples, cancer stem cells may comprise between about 0.5% and 5%, between about 1% and 2.5%, or about 2% of the cell population.

In some examples, cancer associated fibroblast cells may comprise between about 2.5% and 15%, or between about 5% and 10% of the cell population. In some examples, cancer associated fibroblast cells may comprise between about 2.5% and 7.5% of the cell population.

In some examples, a supportive cell (e.g. astrocytes), where present, may comprise between about 0% and 30%, between about 10% and 20%, or about 15% of the cell population.

In some examples, a specialized cell to support a specific microenvironment (e.g. microglia), where present, may comprise between about 10% and 50%, between about 20% and 40%, between about 25% and 30%, or about 28% of the cell population. In some examples, a specialized cell type (such as an adipocyte cell, e.g. a human adipocyte cell), where present, may comprise between about 10% and 30%, such as between about 20% and 25%, or about 23% of the cell population.

In some examples, the construct may comprise or be formed from a population of cells comprising:
(a) about 60% cancer cells (e.g. glioblastoma cells);
(b) about 2% cancer stem cells (e.g. glioblastoma cancer stem cells);
(c) about 10% cancer associated fibroblast cells (e.g. glioblastoma cancer associated fibroblast cells); and
(e) about 28% of a specialized cell type to provide a specific microenvironment (e.g. microglia).

In another example, the construct may comprise or be formed from a population of cells comprising:
(a) about 50% cancer cells (e.g. glioblastoma cells);
(b) about 2% cancer stem cells (e.g. glioblastoma cancer stem cells);
(c) about 5% cancer associated fibroblast cells (e.g. glioblastoma cancer associated fibroblast cells);
(d) about 15% supportive cells (e.g. astrocytes); and
(e) about 28% of a specialized cell type to provide a specific microenvironment (e.g. microglia).

In some examples, the construct may comprise or be formed from a population of cells comprising:
(a) about 70% cancer cells (e.g. breast cancer cells, such as triple negative breast cancer cells);
(b) about 2% cancer stem cells (e.g. breast cancer stem cells);
(c) about 5% cancer associated fibroblast cells (e.g. breast cancer associated fibroblast cells); and
(d) about 23% of a specialized cell type (e.g. adipocyte cells, such as human adipocyte cells).

The extracellular matrix may be selected from any suitable material that can be extruded in the deposition/printing step, that is biocompatible with the cells present in the construct and/or that can support the growth and/or development of the construct.

Suitable matrix materials may include but are not limited to hydrogels, naturally occurring polymers and synthetic polymers. The matrix may be or comprise a hydrogel, for example, a collagen, gelatin, fibrin, polyethylene and/or polysaccharide (e.g. hyaluronic acid, agarose, chitosan, or alginate)-based hydrogel.

Suitable cancer cell compatible materials (based on hydrogels) may include those outlined in the table below.

| BioInk | Material |
|---|---|
| Collagen PREMIUM | Collagen type I |
| Chitoink | Chitosan |
| ColMa | methacrylated collagen |
| Cell-link Laminink | Laminins-basal lining of cell |
| | membrane |
| GelXA laminink | Same as above but more |
| | stable at room temp |
| GelXG | Gelatin-based with Xanthan |
| | gum |
| Alginate | Alginate |
| GelMA High | gelatin methacrylate |
| GelMA-Alginate | gelatin methacrylate |
| GelMA HA | gelatin methacrylate |
| Gel XA | GelMA base, xanthan gum |
| | and alginate |
| Coll 1 | Collagen type I |
| GelMA A | methacrylated collagen and |
| | alginate |
| GelMA C | |
| Cellink A | Sodium alginate |
| Celllink RGD | Polysaccharide hydrogel and |
| | RGD |
| Cellink A RGD | Sodium alginate and RGD |
| Cellink bioink | Polysaccharide hydrogel |

In certain examples, the matrix may be or comprise one or more of: an alginate-based material (e.g. sodium alginate), cellulose (e.g. nanofibrilar cellulose); and extracellular proteins (e.g. laminin). A representative example of a suitable extracellular matrix is Celink Laminink 411 (obtained from Celink LifeSciences). The composition of Laminink 411 is set out below.

| Chemical name | CAS# | EC No. | EC Class |
|---|---|---|---|
| Nanofibrillar | 9004-34-6 | None | Not classified as hazardous |
| Cellulose | | | |
| Sodium Alginate | 9005-38-3 | None | Not classified as hazardous |
| D-mannitol | 69-65-8 | 200-711-8 | Not classified as hazardous |
| Laminin 411 | N/A | None | Not classified as hazardous |
| HEPES buffer | 7365-45-9 | 230-907-9 | Not classified as hazardous |
| solution | | | |

A further example of a suitable extracellular matrix may be GrowInk™ (obtained from UPM Biomedicals), a hydrogel based matrix comprising nanofibrillar cellulose and optionally alginate.

In some examples, the matrix may be a decellularized extracellular matrix material. By way of example, the decellularized extracellular matrix material may be derived from a patient.

The constructs described herein may be prepared and/or obtained by a deposition (e.g. printing) method. As such, according to a further aspect of the disclosure, there is provided a method of making a construct for mimicking an in vivo environment, such as an in vivo cancer microenvironment. The method may comprise:

depositing on a surface:
(a) a cancer cell;
(b) a cancer stem cell; and
(c) a cancer associated fibroblast cell.

In addition to the cell types (a) to (c) noted above, the methods described herein may further comprise depositing one or more of the following:
(e) a supportive cell;
(f) a specialized cell type to provide a specific microenvironment;
(g) an immune cell; and/or
(h) an endothelial cell.

In some examples, the method may further comprise depositing cells (a) to (c) (with optionally one or more of cells (e) to (h)) together with (d) an extracellular matrix.

The step of depositing may be carried out by any suitable technique that facilitates the placement of the components (a) to (c) (and optionally (d) to (h)) at a precise location. Representative examples include, but are not limited to, printing (e.g. bioprinting), spreading, pipetting, spraying, or coating on to the surface. In some examples, the step of depositing comprises printing (e.g. bioprinting) the cells and the extracellular matrix on to the surface.

As such, there is provided a method of making a construct (e.g. a three dimensional construct) for mimicking an in vivo environment, such as an in vivo cancer microenvironment, the method comprising printing on a surface at least one of each of the following:
(a) a cancer cell;
(b) a cancer stem cell; and
(c) a cancer associated fibroblast cell
   to thereby provide the construct.

As used herein, printing may refer to the three dimensional printing of biological material(s). Printing (or bioprinting) may comprise any suitable technique to deposit the components (a) to (c) in or at the desired location on a surface.

The printing may comprise an extrusion based printing technique. By way of example only, the described methods may employ pneumatic printing (which involves extruding a material using air pressure) or may employ a syringe printhead. Printing using a syringe printhead involves mechanically applied pressure to a syringe plunger. The use of a syringe printhead may be helpful to increase consistency of droplet size when working at much smaller volumes, but it can be more challenging to control the shear stress experienced by the cells.

The printing step may comprise printing a plurality of constructs at a series of defined and/or discrete locations on a surface e.g. in a predetermined pattern to provide an array or microarray of constructs. By way of example only, the method may comprise depositing or printing the bioink in a plurality of wells on a multi-well plate, e.g. a 96-well plate or a 384-well plate.

In any of the above described methods and constructs, the various components (a) to (c) (and optionally (d) to (h)) are deposited (e.g. printed) onto the surface to provide the construct. The surface may define a series of locations (e.g. wells) upon which the components may be deposited. Suitable surfaces may be any surface that is compatible (e.g. biocompatible) with the components, is chemically inert and/or that facilitates the culture and/or growth of the construct after deposition. The surface may be or comprise glass, plastic and/or a polymeric material. In some examples, the surface may be or comprise PDMS (polydimethylsiloxane), polycarbonate, or the like.

In particular, the methods disclosed herein may comprise printing droplets of a bioink on to the surface. Within the context of the present disclosure, the bioink may be considered to be made up of the cells (e.g. cells (a) to (c) and optionally one or more of cells (e) to (h)) that have optionally been pre-mixed with and/or are suspended in the extracellular matrix (component (d)).

As used herein, a bioink may be a composition that has suitable rheological, mechanical, and biological properties that facilitate its use in a three dimensional printing method. By way of example, the bioink may be biocompatible, may facilitate mixing, may be extrudable and/or may support cell growth and/or development. Such bio-inks may mimic the extracellular matrix environment and provide support for cell functions such as adhesion, proliferation, and differentiation after printing. It should be appreciated that bio-ink formulations may vary depending on the cancer stem cell type.

The method may comprise culturing cells (a), (b), (c) and optionally any one or more of cells (e) to (h) to provide spheroids prior to the deposition or printing step. In particular, the method may comprise co-culturing cells (a) to (c) (and optionally any one or more of cells (e) to (h)) to provide spheroids prior to the deposition or printing step. Thus, the described methods may involve depositing or printing spheroids on to the surface.

The use of multicellular spheroids (composed of the three or more cell types discussed here) in the deposition or printing step may assist in making the resultant constructs more complex, dense and closer to the in vivo cancer microenvironment.

Alternatively, the method may comprise depositing or printing cells on to the surface. Such methods may be referred to as a “single cell” printing methods herein. Single cell printing methods may offer a particularly consistent method of providing the constructs and/or may provide constructs of greater consistency.

Where the method comprises depositing or printing cells on to the surface, spheroids may form in situ in the deposits. For example, the cells within the deposits may be cultured for a period of time and/or in a suitable culture medium to facilitate the formation of spheroids (e.g. three-dimensional spheroids).

In other examples, the method may comprise depositing or printing a mixture of spheroids and single cells on to the surface.

The use of spheroids within the deposited or printed constructs (whether these are formed before or after the deposition/printing step) can assist in providing a more accurate representation of the in vivo cancer microenvironment as they facilitate cell-cell interactions and/or cell-ECM interactions in a three-dimensional structure.

As used herein, a spheroid (or three-dimensional spheroid) may refer to a three-dimensional cellular aggregate. In some cases, these aggregates may be generally spherical in nature. In a typical two-dimensional monolayer cell culture, cells tend to interact with the substrate upon which they are cultured. In contrast, in a spheroid, cells may be able to grow and/or interact with their surroundings in all three directions and/or a spheroid may enhance cell to cell interactions.

Any suitable technique may be employed for the production of the spheroids as described herein. Representative examples include, but are not limited to, the use of low cell adhesion plates (e.g. where the spheroids form in the rounded bottoms of multi-well plates) and the hanging drop method (e.g. where spheroids form in drops that hang from the surface of a cell plate).

By way of example only, the spheroids may be produced by co-culturing the cells (a) to (c) and optionally (e) to (h) in a low adhesion plate for a pre-determined period of time (e.g. from 0 days to 30 days, such as from 1 day to 21 days, or from 2 days to 14 days) prior to deposition or printing. The cells may be cultured with a medium that promotes the formation of a spheroid (or 3D spheroid). By way of example only, the medium may comprise 3D Tumorsphere medium (obtained from PromoCell, GmbH).

In some examples, the described methods may further comprise a step of magnetic bioprinting.

As used herein, magnetic bioprinting may refer to a printing technique which involves the use of magnetic nanoparticles to print cells into a three-dimensional structure or pattern. In such methods, cells may be tagged with magnetic nanoparticles. External magnetic forces may then be applied to print the cells into a desired three-dimensional structure. Representative examples of magnetic nanoparticles include iron oxides (such as particles consisting essentially of gold, iron oxide and poly-L-lysine e.g. NanoShuttle-PL obtained from Greiner Bio-One).

Magnetic bioprinting may be used to print the cells into spheroids prior to a printing or deposition step as described herein. Additionally or alternatively, magnetic bioprinting may be used to print cells into three-dimensional spheroids after deposition or printing of the bioink on to the surface.

The combination of extrusion based printing and magnetic based bioprinting can assist in providing constructs that mimic an in vivo cancer microenvironment more rapidly and/or can provide a construct that more closely resembles a clinical sample.

In any of the described methods, the cells (a), (b), (c), and optionally any one of more of cells (e), (f) and (h), and/or the spheroids (if used) may be mixed with the extracellular matrix (d) prior to the deposition or printing step. In other words, prior to the printing or deposition step, the cells and/or spheroids may be mixed with the extracellular matrix to provide the bioink formulation.

Following the deposition or printing step, the method may further comprise cross-linking the constructs. The cross-linking step may be useful to provide a degree of rigidity and/or stiffness to the construct. The degree of rigidity and/or stiffness may depend on the nature of the cancer microenvironment that is being mimicked by the construct.

The cross-linking step may be carried out using methods known in the art. In some examples, the cross-linking may be carried out using a cross-linking agent. Suitable cross-linking agents may include metal salts (e.g. calcium chloride). In some examples, the cross-linking step may be light (UV or visible) dependent and/or temperature dependent.

Following the deposition step, the method may then comprise culturing the construct for a period of time such that the construct mimics an in vivo cancer microenvironment. In particular, the construct may be cultured in a medium and/or for a period of time to allow the cells to grow and/or develop so that the construct more closely reflects an in vivo cancer microenvironment. At this stage, the construct may be considered ready for use as a cancer model e.g. in drug testing or development.

In some examples, the construct may be cultured for a period of time anywhere between 1 day and 30 days, in other examples, the construct may be cultured for a period of time anywhere between 2 days and 21 days, or between 3 days and 18 days. In some examples, the construct may be cultured for a period of time between 14 days and 21 days.

The construct may be cultured in any suitable medium (e.g. a culture medium) that promotes growth and/or development of the three-dimensional construct.

The culture medium may comprise growth factors, developmental factors, supplements, buffers and/or other components to promote the growth and/or development of the construct such that it mimics an in vivo cancer microenvironment. By way of example only, the medium may comprise 3D Tumorsphere medium (obtained from PromoCell, GmbH). Alternatively or additionally, the medium may comprise fetal bovine serum solution (e.g FBS replacement solution (Hyclone FBS)), ascorbic acid and/or epidermal growth factor (EGF) growth factor. By way of representative example only, a culture medium may comprise 30 μl Hyclone FBS, 25 ng/ml epidermal growth factor (EGF), and 50 μg/ml ascorbic acid (AA).

The cells and/or spheroids may be mixed with the culture medium prior to the deposition/printing step. In particular, the cells and/or spheroids may be suspended in the culture medium to provide a suspension of cells and/or spheroids prior to the deposition or printing step. In those cases where an extracellular matrix (component (d) is present), the method may comprising mixing the suspension of cells and/or spheroids with the extracellular matrix to provide a bioink formulation that is used in the printing step.

Within the bioink formulation, the relative proportions of the suspension of cells and/or spheroids and the extracellular matrix may be carefully controlled to facilitate the deposition and/or printing step. The relative proportions of the suspension of cells and/or spheroids and the extracellular matrix may be selected dependent upon on the particular cancer environment that is being mimicked by the construct.

In some examples, between about 10% and 90% of the total volume of the bioink formulation is comprised of the suspension of cells and/or spheroids. In some examples, between about 40% and 80% of the total volume of the bioink formulation is comprised of the suspension of cells and/or spheroids. In certain cases, between about 45% and 75% of the total volume of the bioink formulation is comprised of the suspension of cells and/or spheroids.

In some examples, between about 90% and 10% of the total volume of the bioink formulation is comprised of the extracellular matrix. In some examples, between about 60% and 20% of the total volume of the bioink formulation is comprised of the extracellular matrix. In certain cases, between about 55% and 25% of the total volume of the bioink formulation is comprised of the extracellular matrix.

In some examples, between about 40% and 80% of the total volume of the bioink formulation is comprised of the suspension of cells and/or spheroids and between about 60% and 20% of the total volume of the bioink formulation is comprised of the extracellular matrix. The use of such ratios can enhance and/or promote cell proliferation within the constructs and/or can provide stable constructs.

In one case, the bioink formulation may comprise approximately 70% by volume of the suspension of cells and/or spheroids and approximately 30% by volume of the extracellular matrix. In another example, the bioink formulation may comprise approximately 50% by volume of the suspension of cells and/or spheroids and approximately 50% by volume of the extracellular matrix.

In view of the above, according to a yet further aspect of the disclosure, there is provided a bioink formulation for use in making a construct as described herein or for use in a method as described herein.

The bioink formulation may comprise the core cell types (a) to (c) as described herein, optionally together with one or more of cell types (e) to (h) and further optionally together with the extracellular matrix (d) (again as described herein). The relative proportions and properties of the components (a) to (h) that are present in these bioink formulations are described herein in relation to the construct. However, it will be appreciated that the bioink formulation may be in a form that is extrudable and/or that is suitable for a deposition or printing step.

According to another aspect of the present disclosure, there is provided a use of the described constructs for drug development, drug screening and/or clinical evaluation of a drug product.

In particular, an active agent may be applied to and/or contacted with the described constructs which are designed to mimic an in vivo microenvironment (e.g. an in vivo cancer microenvironment). Characteristics of the in vivo cancer microenvironment may be monitored to assess the effect and/or activity of a compound. For example, cell proliferation and/or cell viability may be monitored in the in vivo cancer microenvironment to determine the efficacy of a compound.

The constructs of this disclosure may be used to permit the testing of agents, for example drugs, on cells. The constructs allow a user to monitor and determine the response of a cell to a test agent or drug. An advantage of the constructs disclosed herein is that by maintaining cells in (micro) environments/niches which replicate aspects of the in vivo (micro) environments/niches, the cells will respond to the test agents in a way that better represents how the cells might respond to those test agents/drugs in vivo.

Accordingly, the disclosure provides a method for testing the effects of a test agent or drug on a cell, for example a cancer stem cell, said method comprising, providing a construct of this disclosure, maintaining a cell (e.g. CSC) within said construct, contacting the cell with a test agent and determining the response of the cell (e.g. CSC) to the test agent.

According to a further aspect of the present disclosure, there is provided a kit comprising the core cell types (a) to (c), optionally together with one or more of cell types (e) to (h) and further optionally together with the extracellular matrix (d) (as described herein). These cell types may be supplied and/or stored separately within the kit. Alternatively, the various components may be provided in the kit as a bioink formulation as described herein.

The kit may further comprise one or more of: a culture medium, instructions for use; and a substrate defining a deposition or print surface.

It should be noted that throughout this specification the term “comprising” is used to denote that embodiments of the disclosure “comprise” the noted features and as such, may also include other features. However, in the context of this disclosure, the term “comprising” may also encompass embodiments in which the disclosure “consists essentially of” the relevant features or “consists of” the relevant features.

### DETAILED DESCRIPTION

The disclosure will now be further described, by way of example only, with reference to the following Figures.

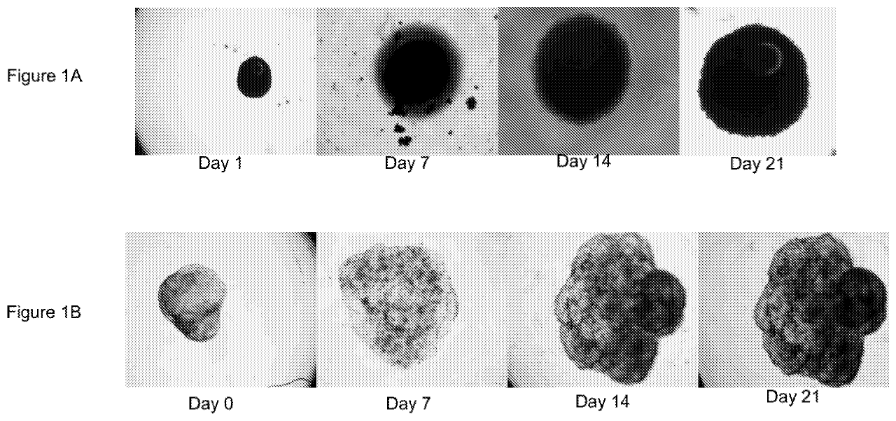

FIG. 1A shows brightfield images taken of a 3D spheroid model set up at the same time to the bioprinted models (as disclosed herein) to provide a comparison. The 3D spheroid models have two cell types (cancer cells and cancer associated fibroblasts) with no ECM. As shown in this figure, they do not represent the heterogeneity of the cancer, and take up to 60-90 days to be ready for drug discovery assays.

FIG. 1B shows the brightfield images taken of bioprinted construct according to an example B21_012 of the present disclosure (sometimes referred to herein as a 3D CarcinoGBM™ model). This consists of five different cell types in an ECM based bioink, the entire tumour is bioprinted using bioprinters. The images shown in the Figure are taken of the same well with Day 0 representing the day the tumour was bioprinted to day 21 of growth. The bioprinted mini tumour has cell proliferation at a rate akin to the microenvironment and the constructs become more dense. As compared to the above 3D spheroid images of Glioblastoma cells (exemplified in FIG. 1A), the exemplified model shows increased complexity, is closer to the clinical tumour as seen in literature and has more cell types involved, which encapsulates the heterogeneity of the cancer in an accurate, rapid manner than a homogenous 3D spheroid model. The model shown in FIG. 1B may be ready for use in a drug discovery assay by day 14.

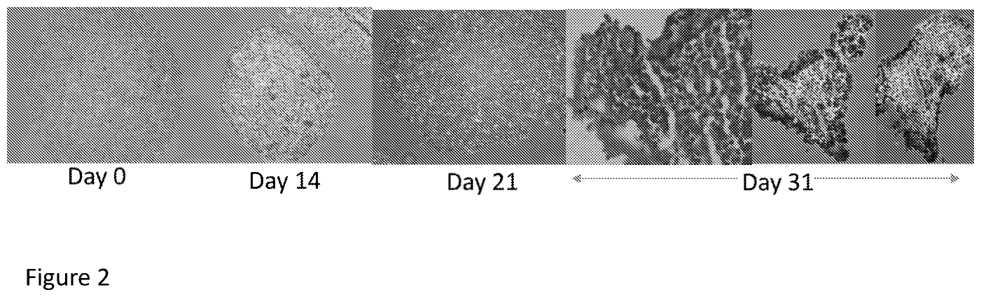

FIG. 2 above shows the hematoxylin and eosin stain (often abbreviated as: H&E stain or HE stain) of a glioblastoma printed tumour model according to an example B21_012 of this disclosure. The figure shows the growth of the glioblastoma printed tumour, bioprinted at Day 0, with the constructs being ready for drug testing at between Day14-21. The growth, different cell types and tumour stroma and ECM can be easily seen in these images, with invasion/migration also observed. Compared to literature, the inventors have observed that the bioprinted tumour model of this example is very close to the clinical biopsy samples giving strong validation data.

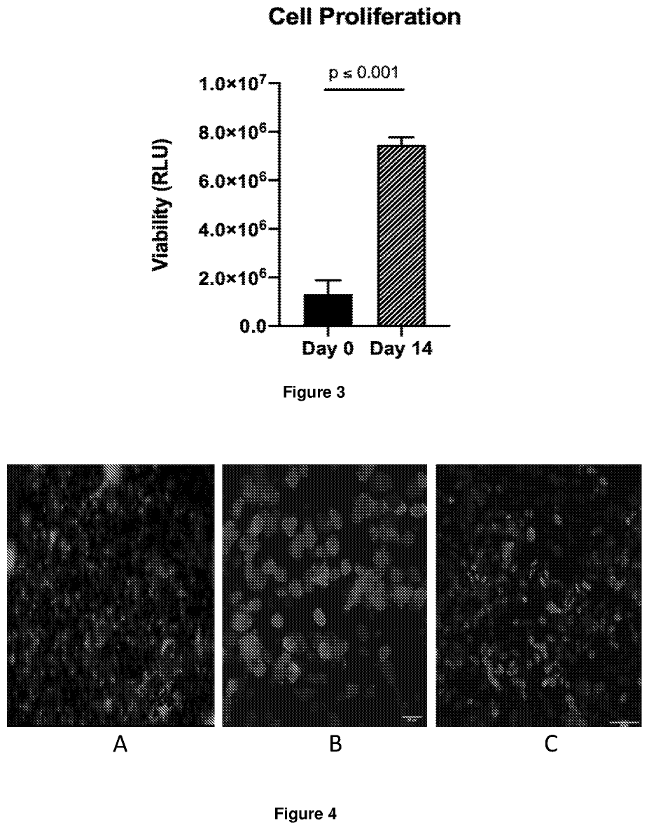

FIG. 3 shows the results of a cell viability studies of a bioprinted construct according to an example (B21_012) of the disclosure. Day 0 corresponds to the day the construct was bioprinted and Day 14 is 14 days after bioprinting. As can be seen from the results in the Figure, the Day 14 results for the model is much higher than Day 0 indicating proliferation, high cell viability and growth. This indicates that the 3D bioprinted model is ready for drug screening and testing 14-21 days after bioprinting, a much shorter, rapid timeframe as compared to existing 3D organoid and spheroid solutions which take a minimum of 60 days to 90 days to be ready for drug screening.

FIG. 4 shows phenotypic characterisation of an example construct according to example B21_012 of this disclosure. There are various antibody markers that are expressed by Glioblastoma tumour tissues and this is known in literature and peer-reviewed papers. For phenotypic characterisation of the constructs, immunostaining was performed using certain markers expressed only in 3D. These studies confirmed that a 3D microenvironment had been established in the example construct.

In particular:

FIG. 4A shows an example bioprinted tumour construct stained with anti-CD133-SB436 (detects CD133 marker on human cells) which is a stem cell marker. The role of the cell surface CD133 as a cancer stem cell marker in glioblastoma (GBM) has been widely investigated, since it identifies cells that are able to initiate neurosphere growth and form heterogeneous tumors. (Paola Brescia. et al, 2013). As can be seen from the figure, the construct is positive for CD133 (red fluorescence), previously not observed in 2D cells and matches clinical tumour data.

FIG. 4B shows an example bioprinted tumour construct stained with Alexa Fluor® 594 Anti-CD44 antibody (detects CD44 marker on human cells). CD44 promotes GBM aggressiveness by increasing tumor cell invasion, proliferation and resistance to standard chemoradiation therapy. CD44 is a transmembrane molecule overexpressed in GBM. Targeting CD44 is a promising GBM therapy. (Kelly L. Mooney, et al, 2016). This figure shows CD44 positive 3D GBM microenvironment (red fluorescence) counterstained with nuclei dye Hoechst 33342 and matches clinical tumour data.

FIG. 4C shows an example bioprinted tumour construct stained with Anti-Nestin-A488 (detects Nestin marker on human cells). Nestin is one of the intermediate filaments abundantly produced in the developing central nervous system. Nestin is also detected in gliomas/glioblastomas. Nestin is not only a marker for neuroepithelial stem cells and glioma cells but also for tumor endothelial cells during rapid growth (Sugawara, Ki., Kurihara, H., Negishi, M. et al, 2002). As can be seen from the FIG. 4C, the model is positive for Nestin (green fluorescence), previously not observed in 2D cells and matches clinical tumour data.

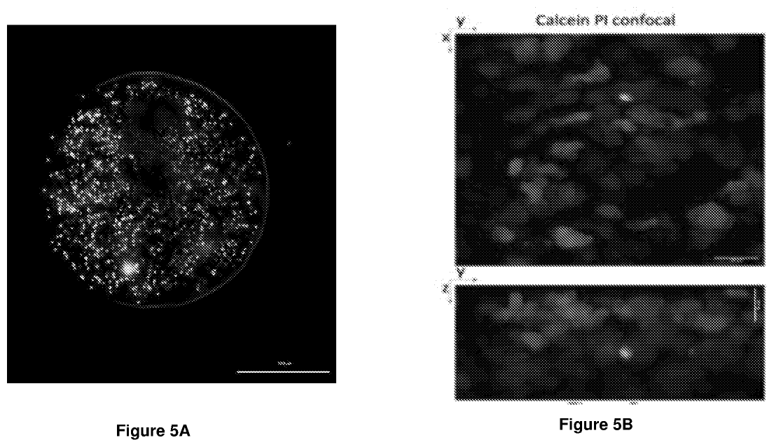

FIG. 5 shows phenotypic characterisation of an example construct according to example B21_012 of this disclosure. The biomarkers in our models were characterized using immunofluorescence techniques that stained both live and fixed samples with florescence tagged antibodies.

In particular:

FIG. 5A shows cell viability of an example bioprinted construct using Calcein-AM assay. During the Calcein AM assay, hydrolysis of Calcein AM by intracellular esterases produces a hydrophilic, strongly fluorescent compound that is retained in the cell cytoplasm and can be measured at Ex/Em=485/530 nm. The measured fluorescence intensity (green) is proportional to the number of viable cells. As can be seen in this figure, the example construct had a high cell viability. The image was taken live using fluorescent microscope in-house.

FIG. 5B shows cell viability of an example bioprinted construct using Calcein-AM assay alongside Propidium iodide (PI). PI is a popular red-fluorescent nuclear and chromosome counterstain. Since propidium iodide is not permeant to live cells, it is also commonly used to detect dead cells in a population. PI binds to DNA by intercalating between the bases with little or no sequence preference. A combination of Calcein-AM and PI detects live and dead cells and as can be seen from this image, a high number of live cells, and negligible dead cells were observed in the example construct. The constructs are counterstained with Hoechst 33342. The Hoechst stains are a family of blue fluorescent stains for labelling DNA in fluorescence microscopy. Because these fluorescent stains label DNA, they are also commonly used to visualize nuclei and mitochondria.

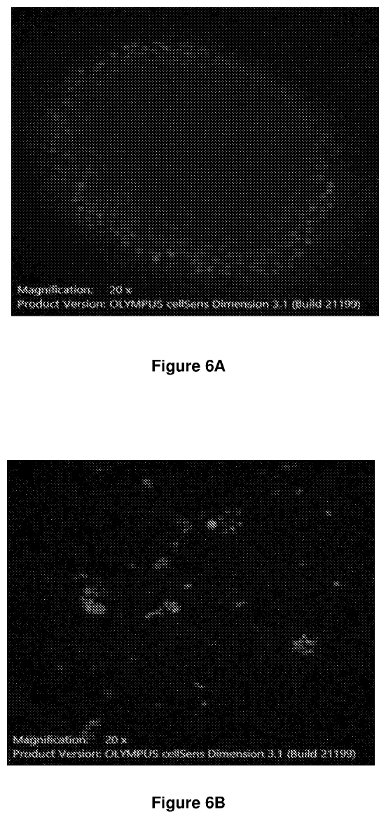

FIG. 6 shows phenotypic characterisation of an example construct according to example B21_012 of this disclosure.

In particular:

FIG. 6A shows an example bioprinted tumour construct stained with Anti-EphA2. EphA2 is both specifically overexpressed in GBM and expressed differentially with respect to its ligand, ephrinA1, which may reflect on the oncogenic processes of malignant glioma cells. EphA2 seems to be functionally important in GBM cells and thus may play an important role in GBM pathogenesis. Hence, EphA2 represents a new marker and novel target for the development of molecular therapeutics against GBM. (Wykosky J. et al, 2005)

As can be seen from the figure, the example construct is positive for EphA2 (blue fluorescence).

FIG. 6B shows an example bioprinted tumour construct stained with Alexa Fluor® 568 Anti-GFAP antibody (detects GFAP marker on human cells). Glial fibrillary acidic protein (GFAP), first described in 1971 by Eng et al., is a member of the cytoskeletal protein family and is widely expressed in astroglial cells. GBM tumour tissue samples showed a strong variability in GFAP expression ranging from 25% in some patients to almost 100% in others. (C. S. Jung. et al, 2007). This matches the 3D printed tumour phenotypic results and the example constructs express GFAP in 3D rather than 2D. The FIG. 6B shows GFAP positive 3D printed tissue (red fluorescence) counterstained with nuclei dye Hoechst 33342.

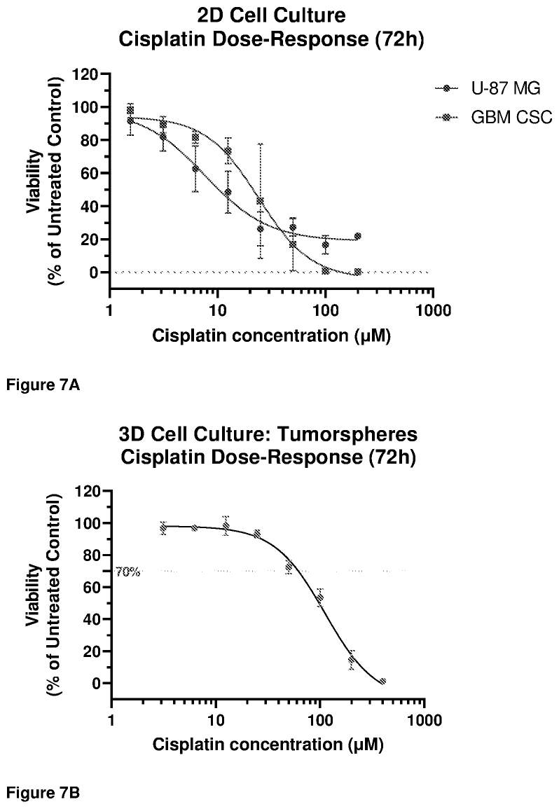

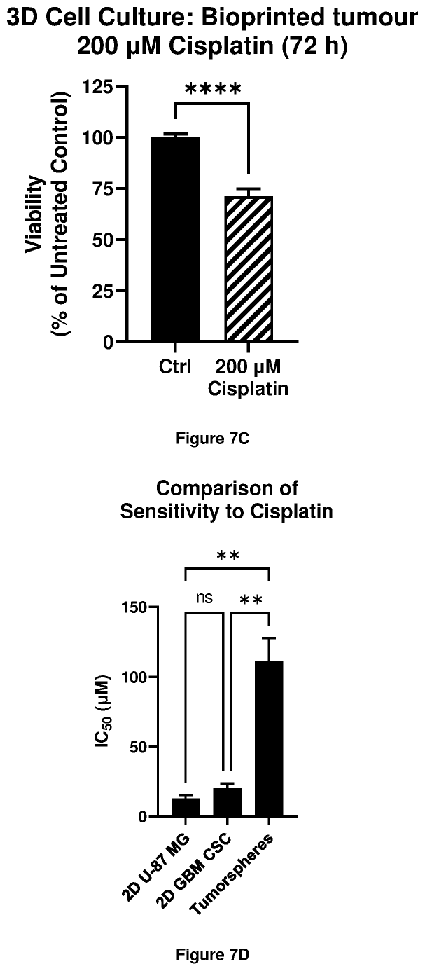

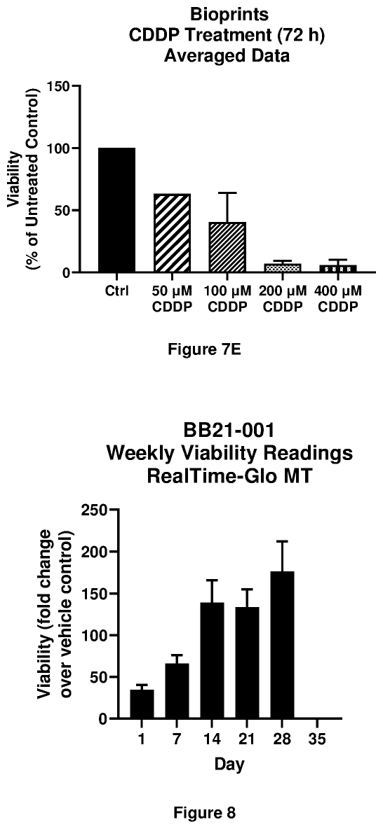

FIG. 7 shows the response of 2D, 3D and 3D bioprinted GBM cell cultures to cisplatin.

FIG. 7A shows cisplatin dose-response in 2D cultures of glioma cell line (U-87 MG) and cancer stem cells (GBM CSC).

FIG. 7B shows cisplatin dose-response in 3D GBM tumorspheres (spheroids) derived from a coculture of U-87 MG, GBM CSC, GBM CAF and microglia (HMC3).

FIG. 7C shows treatment of 3D GBM bioprints (made in accordance with example B21_012) with 200 μM cisplatin.

Cell cultures were exposed to the indicated cisplatin concentrations for 72 h. Cell viability was then assessed using the luminescence-based CellTiter-Glo Assay. Data are presented as means±SEM (n≥2). For 2D cell cultures and 3D tumorspheres FIGS. 7A and 7B) dose-response curves were fitted (nonlinear regression) to calculate the IC₅₀ values (see: Table 1 in the examples section). In the 3D bioprint experiment (FIG. 7B) statistically significant difference between untreated control and cisplatin treatment was determined using an unpaired t-test (**** p≤0.0001).

FIG. 7D shows a comparison of cisplatin IC₅₀ values in 2D and 3D GBM cell cultures. Data are presented as means±SEM (n≥2). Statistically significant differences between the IC₅₀ values determined using one-way ANOVA with Tukey's post-hoc test and indicated as follows: ** p≤0.01, ns—not significant.

FIG. 7E shows cisplatin treatment of bioprints (made in accordance with example B21_012). Bioprinted constructs were treated with 50, 100, 200, or 400 μM cisplatin (CDDP) for 72 h. Viability was assessed using CellTiter-Glo assay.

FIG. 8 shows cell viability of an example bioprinted construct (BB21-001). Viability was assessed using RealTime-Glo MT. Data presented as mean±SD, n is 20 (where n represents the total number of constructs tested)

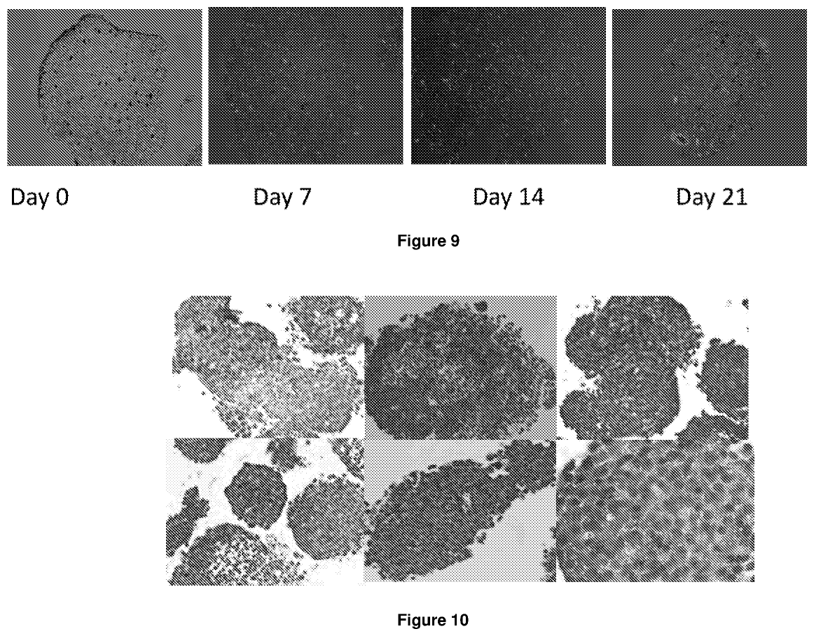

FIG. 9 shows histology images, in particular the hematoxylin and eosin (H&E) stain of a breast cancer printed tumour model according to an example BB21-001 of this disclosure. The figure shows the growth of the breast cancer printed tumour, bioprinted at Day 0 and with the constructs being ready for drug testing at between Day 14-21. The growth, different cell types and tumour stroma and extracellular matrix (ECM) can be easily seen in these images, with invasion/migration also observed. Compared to literature, the inventors have observed that the bioprinted tumour model of this example is very close to the clinical biopsy samples giving strong validation data.

FIG. 10 shows further images of the hematoxylin and eosin (H&E) stain of a breast cancer (triple negative) printed tumour model according to an example BB21-001 of this disclosure. BB21-001 printed with four different cell types and ECM (extracellular matrix) that matches the breast cancer microenvironment. The inventors have observed that the H&E images closely correspond to the tumour biopsy H&E observed in the literature (see, for example, Pareja et al, “Triple-negative breast cancer: the importance of molecular and histologic subtyping, and recognition of low-grade variants”, npj Breast Cancer, 2, 16036 (2016)).

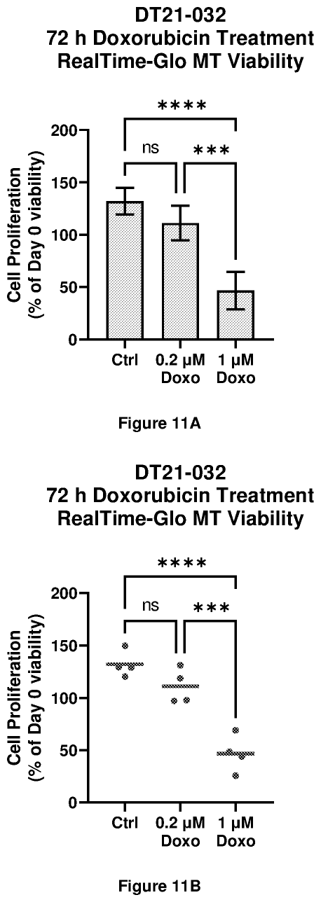

FIG. 11 shows doxorubicin treatment of single-cell bioprint made in accordance with example BB21-001 of this disclosure. The 15-day old bioprinted constructs were treated with 0.2 and 1 μM Doxorubicin (Doxo) for 72 h. Cell viability was assessed using RealTime-Glo MT before treatment (day 0) and after 72 h of treatment. To assess the doxorubicin effects after 72 h, a percentage of day 0 viability was calculated for each construct. The results are shown in FIGS. 11A and 11B. Statistically significant differences were determined using one-way ANOVA with Tukey's post-hoc test, and indicated as follows: *** p≤0.001, **** p≤0.0001, ns—not significant. FIG. 11A shows the data presented as means±SD, n=4; and FIG. 11B shows a scatter plot of the same data.

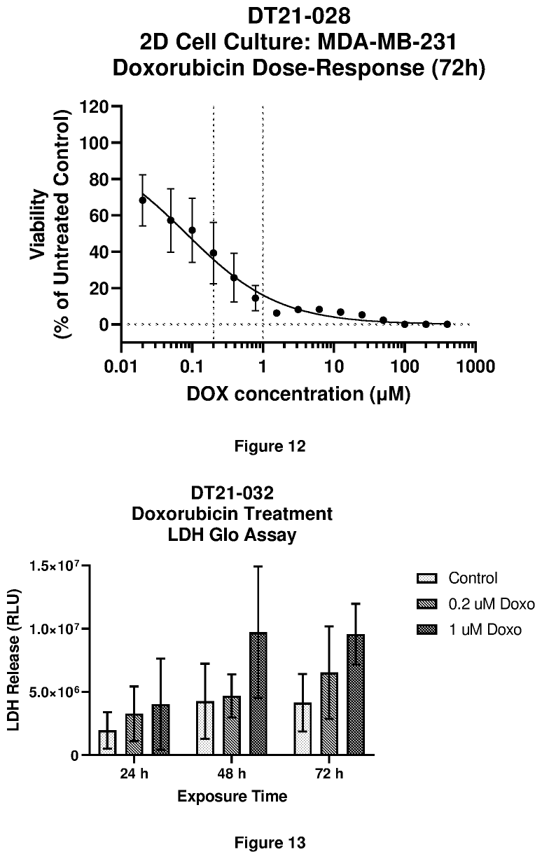

FIG. 12 shows a dose response curve for a 2D cell culture of MDA-MB-231 treated with doxorubicin over a period of 72 hours. The MDA-MB-231 cells were cultured in Leibovitz's L-15 Medium (ATCC®) and fetal bovine serum was added to a final concentration of 10%, and in accordance with the general procedure outlined in MDA-MB-231 (HTB-26™) Product sheet from ATCC® (https://www.atcc.org/products/htb-26).

FIG. 13 shows doxorubicin treatment of single-cell bioprinted construct according to example BB21-001 of this disclosure. The 15-day old bioprinted constructs were treated with 0.2 and 1 μM Doxorubicin (Doxo). Medium samples were collected at 24, 48 and 72 h time points to assess the Lactate Dehydrogenase (LDH) release, which is a marker of cytotoxicity. LDH-Glo assay was used. Data are presented as means±SD, n=3.

### EXAMPLE METHODS

### General Manufacturing Method

The production of each construct uses an extrusion-based 3D bioprinter (BioX from CELLINK).

1. Cancer cells, cancer stem cells and cancer associated fibroblasts (and any other cells that are used) are co-cultured together in a predetermined ratio (specific ratios disclosed in the following examples). These are co-cultured in 96 well low adhesion plates to form 3D spheroids

2. These 3D spheroids are fed with a special 3D Tumorsphere medium supplied by a company called PromoCell.

3. These conditions enable the 3D spheroids to form a uniform shape per well with the three cell types. These are cultured for 14 days before bioprinting them into microenvironment conditions.

In some cases, the cells are tagged with magnetic nanoparticles which allows the generation of 3D spheroids via magnetic bioprinting. In such cases, the 3D spheroids can be ready for bioprinting within 5-9 days as compared to 14 days.

4. After the 3D spheroid generation with the different cell types, the spheroids are mixed with a bioink based on alginate optionally comprising laminin proteins (e.g. CELLINK Laminink 411, CELLINK Laminink plus and CELLINK RGD-A). The Laminin based bioinks may offer a good structure to the microenvironment and allow cells to grow in them. The 3D spheroids and the bioinks are mixed together using syringes to form a uniform bioink plus living cells spheroid mixture for bioprinting.

5. The printing step is carried out using a syringe printhead in an extrusion-based printer. The parameters are controlled to control the size of droplets printed per well. Typically, 3-5 μl size droplets are printed per well of a 96 well plate. The printer prints the entire plate in 5 minutes, offering great speed for generation of multiple plates for volume manufacturing.

6. After printing, calcium chloride solution may be used to cross-link each droplet.

In some examples, magnetic bioprinting may be carried out at this stage. For example, the cells may be magnetised prior to mixing with the ink and prior to printing into the well. In such an example, a step of magnetic bioprinting may be carried out after printing to generate spheroids quickly within the constructs.

7. The cells are then fed with the 3D tumorsphere medium (PromoCell) and 10% FBS replacement solution (Hyclone FBS which is animal free) and 25 ng/ml of EGF growth factor may be added. Cell viability checks are done using a Luminescence ATP based assay for 3D structures at time points day 0, 7, 14, 21 and 31. Histology sections using OCT freezing and cryostat sectioning along with staining with H&E stains is performed at day 14, 21, 31 and day 60. The constructs can stay viable for more than 60 days and have a good viability and a biopsy tumour-like (clinical) histology result.

8. The bioprinted constructs can be treated with drugs at either timepoints 21, 31 or 60 days and can be utilised for various assays.

9. For the immuno-oncology model, the same steps (1) to (8) may be carried out but additional cell types may be added (e.g. macrophages). In one example composition, the ratios may be 60% U87MG cells, 28% Microglia, 5% M2 Macrophages, 5% GBM-CAFs and 2% GBM-CSCs.

For the single cell printing methods disclosed herein, the general procedure typically involves mixing each different cell type in the defined ratios and then printing in line with the general printing steps described above in steps (4) to (6) that are described above in relation to the printing of spheroids.

### EXAMPLE CONSTRUCTS

A number of specific example constructs have been prepared in accordance with the general procedure outlined above. The details are provided below.

### Example B21_012

| | | Percentage | Number of tumorspheres |
|---|---|---|---|
| Cell line | Passage | (%) | used |
| U87MG | P2 | 60 | 480 (5 × 96 well plates) |
| GBM CAF | P18 | 10 | |
| GBM CSC | P0 (thawed cells) | 2 | |
| HMC3 | | 28 | |

Tumorspheres, obtained from a coculture of U87MG (60%), microglia HMC3 (28%), GBM-CAF (10%) and GBM-CSC (2%) after 14 days in 5×96-well round bottom low adhesion plates, were harvested by sedimenting in Falcon tube then transferred into a 1 ml syringe. The culture medium was eliminated by gravitation. The total volume of tumorsphere pellet was 50 μL. 270 μL of bioink Cellink Laminink 411 were transferred into another 1 ml syringe; a connector was used to link both syringes. Mixing the bioink and tumorspheres was done by pushes from one side to another. The homogenous mixture tumorspheres-bioink (˜320 μL) was transferred in 3 ml syringe preloaded with 1 mL start ink. Dark green needle (18 G) was fitted to the syringe then set to bioprint.

### Example B21_018

| | | Percentage | Number of tumorspheres |
|---|---|---|---|
| Cell line | Passage | (%) | used |
| U87MG | P23 | 50 | 16.6E6 cells |
| GBM CAF | P18 | 5 | |
| GBM CSC | P23 | 2 | |
| Microglia HMC3 | P16 | 28 | |
| Astrocytes | P5 | 15 | |
| IHACLON4 | | | |

Co-cultured U87MG (50%), microglia HMC3 (28%), Astrocytes-IHACLON4 (15%), GBM-CAF (5%) and GBM-CSC (2%) were mixed, spun down, resuspended in 30 μL of FBS containing 25 ng/ml EGF (105 μL) then loaded into a syringe containing 370 μL of bioink Laminink 411. were transferred into another 1 ml syringe; a connector was used to link both syringes (pictures). Mixing the bioink and tumorspheres was done by pushing from one side to another. The homogenous mixture tumorspheres-bioink (˜320 μL) was transferred in 3 ml syringe preloaded with 1 mL start ink. Dark green needle (18 G) was fitted to the syringe then set to bioprint.

N.B. Astrocytes noted in the following examples are Immortalized Human Astrocytes (IM-HA) obtained from Innoprot of Parque Tecnológico de Bizkaia, Spain).

### Example B21_021

| | | Percentage | Number of tumorspheres |
|---|---|---|---|
| Cell line | Passage | (%) | used |
| U87MG | P28 | 50 | 15E6 cells |
| GBM CAF | P23 | 5 | |
| GBM CSC | P27 | 2 | |
| Microglia HMC3 | P19 | 28 | |
| Astrocytes | P8 | 15 | |
| IHACLON4 | | | |

Co-cultured U87MG (50%), microglia HMC3 (28%), Astrocytes-IHACLON4 (15%), GBM-CAF (5%) and GBM-CSC (2%) were mixed, spun down, resuspended in 30 μL of FBS containing 25 ng/ml EGF (105 μL) then loaded into a syringe containing 370 μL of bioink Laminink 411. were transferred into another 1 ml syringe; a connector was used to link both syringes. Mixing the bioink and tumorspheres was done by pushing from one side to another. The homogenous mixture tumorspheres-bioink (˜320 μL) was transferred in 3 ml syringe preloaded with 1 mL start ink. Dark green needle (18 G) was fitted to the syringe then set to bioprint.

### Example B21_022

| | | Percentage | Number of tumorspheres |
|---|---|---|---|
| Cell line | Passage | (%) | used |
| U87MG | P11 | 50 | 15E6 cells |
| GBM CAF | P30 | 5 | |
| GBM CSC | P29 | 2 | |
| Microglia HMC3 | P19 | 28 | |
| Astrocytes | P8 | 15 | |
| IHACLON4 | | | |

Co-cultured U87MG (50%), microglia HMC3 (28%), Astrocytes-IHACLON4 (15%), GBM-CAF (5%) and GBM-CSC (2%) were mixed, spun down, resuspended in 30 μL of FBS containing 25 ng/ml EGF (105 μL) then loaded into a syringe containing 370 μL of bioink Laminink 411. were transferred into another 1 ml syringe; a connector was used to link both syringes. Mixing the bioink and tumorspheres was done by pushing from one side to another. The homogenous mixture tumorspheres-bioink (˜320 μL) was transferred in 3 ml syringe preloaded with 1 mL start ink. Dark green needle (18 G) was fitted to the syringe then set to bioprint.

### Exemplary Magnetic Bioprinting Method—Example B21_017

| | | | |
|---|---|---|---|
| | | Percentage | Number of tumorspheres |
| Cell line | Passage | (%) | used |

| | | |
|---|---|---|
| U87MG | 60 | Magnetic Bioprinting |
| GBM CAF | 10 | |
| GBM CSC | 2 | |
| HMC3 | 28 | |

U87MG (60%), microglia HMC3 (28%), Astroglia (15%), GBM-CAF (5%) and GBM-CSC (2%) were seeded and magnetised on the next day with different ratios of NanoShuttles (40 μL, 100 μL & 200 μL), harvested on the following day and counted. 1E4 cells (in 100 μL medium) were seeded per well in a 96-well flat bottom cell repellant plate. The plate was placed on the Spheroid Drive and incubated for 1 h (1st check=>spheroid formation in 100 μL & 200 μL but 40 μL no cells). Added cells from the 40 μL NanoShuttle then another hour incubation. The rest of cells (of different ratios) was mixed and bioprinted within GrowInk (405 μL). The homogenous mixture tumorspheres-bioink (˜320 μL) was transferred in 3 ml syringe preloaded with 1 mL start ink. Dark green needle (18 G) was fitted to the syringe then set to bioprint.

### Example B21_019 (Magnetic Bioprinting Used to Form Spheroids Prior to Bioprinting)

| | | Percent- | Number of tumorspheres |
|---|---|---|---|
| Cell line | Passage | age (%) | used |
| U87MG | all 5 cell lines were | 50 | 3.2E6 cells |
| GBM CAF | harvested & seeded | 5 | |
| GBM CSC | at usual proportions | 2 | |
| Microglia | for a total of 1E6 | | |
| HMC3 | cells in a T25, the | | |
| Astrocytes | next day cells were | 28 | |
| IHACLON4 | magnetised with | 15 | |
| | 40 uL of nanoshuttles | | |
| | then incubated for | | |
| | OVN | | |

1E6 cells (Cultured U87MG (50%), microglia HMC3 (28%), Astrocytes-IHACLON4 (15%), GBM-CAF (5%) and GBM-CSC (2%)) were seeded in a T25 (02/06/21), magnetised (using 40 μL NanoShuttles) and then harvested 2 days later. 1E4 cells per well were seeded. Each plate was incubated on the Spheroid Drive to get cells stick together (spheroid formation). Plate 1 was used in testing incorporation Magnetic Bioprint into GrowInk. GrowInk was diluted 50/50, with Hyclone, bioprinted (EV=1.5 uL, RV=10 uL & R=10 uL/s) then spheroids were transferred on top, spun down (1500 rpm, 5 min).

### Example BB21-001 (Exemplary Breast Cancer Model Obtained Via “Single Cell” Printing Method)

| | | Percentage | Number of cells |
|---|---|---|---|
| Cell line | Passage | (%) | used |
| Breast cancer cell | P54 | 70 | 2600000 |
| (MDA-MB-231 (ATCC | | | |
| HTB-26)) | | | |
| Adipocyte cells | P3 | 23 | 845000 |
| (hMAds) | | | |
| Breast cancer | P11 | 5 | 200000 |
| associated fibroblast | | | |
| (CAF) | | | |
| Breast cancer stem | P25 | 2 | 73000 |
| cell (BCSC) | | | |
| | | | 3718000 |

Bioink formulation used: Laminink 411 (200 uL) from Cellink.

Each cell line (MDA-MB-231, hMAds, CAF and BCSC) was harvested, counted and then the required number of cells was transferred into a 15 ml Falcone tube. The cells were spun down then resuspended into 20 μL of complete 3D Tumorsphere Medium XF (obtained from PromCell®) and loaded into a syringe preloaded with 200 μL of Laminink 411 (cells corresponded to 10% of the bioink volume). Then mixed for 550 times. The homogeneous mixture was transferred to a syringe and then set to bioprint (the extrusion volume of each bioprint being set to 3 uL (approximately 50000 cells per droplet).

### Example BB21-002 (Exemplary Breast Cancer Model Obtained Via “Single Cell” Printing Method)

| | | Percentage | Number of cells |
|---|---|---|---|
| Cell line | Passage | (%) | used |
| MDA-MB-231 (ATCC | P54 | 70 | 1220000 |
| HTB-26)) | | | |
| hMAds | P3 | 23 | 400000 |
| CAF | P11 | 5 | 87000 |
| BCSC | P25 | 2 | 34800 |
| | | | 1741800 |

Bioink formulation used: Laminink 411 (200 μL) from Cellink.

Each cell line (MDA-MB-231, hMAds, CAF and BCSC) was harvested, counted and then the required number of cells was transferred into a 15 ml Falcone tube. The cells were spun down then resuspended into 20 μL of complete 3D Tumorsphere Medium XF (obtained from PromoCell®) and loaded into a syringe preloaded with 200 μL of Laminink 411. Then mixed for 550 times. The homogeneous mixture was transferred to a syringe and then set to bioprint (the extrusion volume of each bioprint being set to 3 uL).

### Evaluation of Constructs—Drug Testing (Glioblastoma)

Cisplatin is a chemotherapy medication used to treat a number of cancers. These include testicular cancer, ovarian cancer, cervical cancer, breast cancer, bladder cancer, head and neck cancer, esophageal cancer, lung cancer, mesothelioma, brain tumours and neuroblastoma.

Response to cisplatin was first tested in 2D cultures of U-87 MG glioblastoma cell line and glioblastoma cancer stem cells (see FIG. 7A). Cells were exposed to a range of cisplatin concentrations for 72 hours and sensitivity to this drug was evaluated by determining a concentration that kills 50% of the cells (IC₅₀). The higher the IC₅₀ concentration, the higher the resistance to drug treatment. Both cell lines were deemed sensitive to cisplatin, however, higher drug doses were required to initiate death of cancer stem cells.

In the next step, response to cisplatin was tested in 3D GBM spheroids (see FIG. 7B). As expected, an increased resistance to cisplatin was observed (in comparison to 2D cultures), as 50% reduction in cell viability was achieved at higher drug concentrations. Nevertheless, cisplatin was effective in 3D GBM tumourspheres (spheroids) as well.

Since 50 μm of the Cisplatin (CDDP) dose only killed 30% of cells in the printed constructs of the present disclosure, the dosage was increased in order to determine an IC₅₀ value of cisplatin for the constructs and positive control. When the dosage was increased to 100 μm, the CDDP (cisplatin) only killed 37% of the cells. This meant an even higher dose was required as the printed constructs were resistant to treatment at this dosage. In the previous treatment of 3D spheroids (tumourspheres), the IC₅₀ value was determined to be 111 μm. These results indicate that the presently described 3D printed constructs were much more resistant than the 3D spheroids (tumourspheres) and needed a higher dosage to kill 50% of the cells. In particular, the IC₅₀ value of cisplatin treatment on the printed construct was determined to be higher than 200 μm dosage as can be seen from FIGS. 7C and 7D. This is much higher than 3D spheroid dosage, and closer to a patient sample dosage.

These results indicate an advantage of the constructs described herein when compared to the 3D spheroids commonly used in laboratories. In particular, the results indicate that the printed constructs are closer to a patient biopsy tissue/clinical tissue as seen in literature and was resistant to treatments as would a patient biopsy sample. These results are indicative that the printed model provides an improvement over 3D spheroids/organoids in terms of drug testing, and provides results closer to those that would be observed in the clinic.

The above results are further illustrated in Table 1 below:

| | Cisplatin IC₅₀ |
|---|---|
| Cell Culture | (Mean ± SEM) |
| 2D U-87 MG | 12.9 ± 2.5 μM |
| 2D GBM CSC | 20.1 ± 3.5 μM |
| 3D Tumorspheres | 111.0 ± 16.8 μM |
| 3D Bioprinted tumour | >200 μM |

Table 1 showing cisplatin IC₅₀ values in 2D and 3D GBM cultures.

As can be seen from the table above, constructs made in accordance with the present disclosure provided increased resistance to cisplatin and more closely resemble results that would be obtained with a patient biopsy sample.

### Evaluation of Constructs—Drug Testing (Triple Negative Breast Cancer)

Doxorubicin is a chemotherapy medication used to treat a number of cancers.

Response to doxorubicin was first tested in 2D cultures of triple negative breast cancer cells (MDA-MB-M231) (see FIG. 12). Cells were exposed to a range of doxorubicin concentrations for 72 hours and sensitivity to this drug was evaluated by determining a concentration that kills 50% of the cells (IC₅₀). The higher the IC₅₀ concentration, the higher the resistance to drug treatment. An IC₅₀ of 0.08±0.02 UM was observed for doxorubicin (DOX).

In the next step, response to doxorubicin was tested in an example construct of this disclosure (BB21-001) (see FIGS. 11A and 11B).

The results are illustrated in Table 2 below:

| Concentration of doxorubicin | 2D cell culture of | |
|---|---|---|
| (μM) | MDA-MB-231 | BB21-001 |
| 0.2 | ~40% | ~84% |
| 1.0 | <14% | ~35% |

Table 2 showing cell viability (in comparison to an untreated control sample) of 2D cell culture versus single cell bioprinted construct according to example BB21-001 of the disclosure at (i) 0.2 μM of doxorubicin; and (ii) 1.0 μM of doxorubicin.

The inventors observed that the treatment of the example bioprint constructs according to this disclosure with 1.0 μM doxorubicin significantly reduces cell viability. The inventors further observed that the bioprinted constructs were more resistant to doxorubicin treatment than the model provided by the 2D culture of MDA-MB-231 cells (see, for example, FIG. 12).

Thus, this table provides further evidence that constructs made in accordance with the present disclosure provided increased resistance to doxorubicin and more closely resemble results that would be obtained with a patient biopsy sample.

Cytotoxicity of doxorubicin was also assessed by LDH release detection. The inventors observed an increasing trend at 72 h time-point. This provides further evidence of the utility of the constructs as useful models for testing of drugs.

1. A construct for mimicking an in vivo environment, such as an in vivo cancer environment, the construct comprising at least one of each of the following:
(a) a cancer cell;
(b) a cancer stem cell;
(c) a cancer associated fibroblast cell; and optionally,
(f) a specialized cell type to provide a specific microenvironment.

2. The construct according to claim 1, further comprising one or more of the following:
(e) a supportive cell;
(g) an immune cell; and/or
(h) an endothelial cell.

3. The construct according to claim 1, further comprising an extracellular matrix.

4. The construct according to claim 1, wherein the cancer cell is selected from brain cancer cells, lung cancer cells, breast cancer cells, prostate cancer cells, colorectal cancer cells, ovarian cancer cells, pancreatic cancer cells, skin cancer cells, bone cancer cells and the like.

5. The construct according to claim 1, wherein the cancer stem cell is selected from brain cancer stem cells, lung cancer stem cells, breast cancer stem cells, prostate cancer stem cells, colorectal cancer stem cells, ovarian cancer stem cells, pancreatic cancer stem cells, skin cancer stem cells, bone cancer stem cells and the like.

6. The construct according to claim 1, wherein the cancer associated fibroblast cell is selected from brain cancer associated fibroblast cells, lung cancer associated fibroblast cells, breast cancer associated fibroblast cells, prostate cancer associated fibroblast cells, colorectal cancer associated fibroblast cells, ovarian cancer associated fibroblast cells, pancreatic cancer associated fibroblast cells, skin cancer associated fibroblast cells, bone cancer associated fibroblast cells and the like.

7. The construct according to claim 1, wherein (a) is a glioblastoma, (b) is a glioblastoma cancer stem cell and (c) is a glioblastoma cancer associated fibroblast cell.

8. The construct according to claim 2, wherein the supportive cell is selected from epithelial cells and astrocytes

9. The construct according to claim 1, wherein the specialized cell type to provide a specific microenvironment is selected from a microglial cells and adipocyte cells.

10. The construct according to claim 2, wherein the immune cells are selected from lymphocytes, macrophages and the like.

11. The construct according to claim 1, wherein the construct comprises a glioblastoma, a glioblastoma cancer stem cell, a glioblastoma cancer associated fibroblast cell, an astrocyte and a microglial cell.

12. The construct according to claim 1, wherein the construct comprises a breast cancer cell, a breast cancer stem cell, a breast cancer associated fibroblast cell and an adipocyte cell (such as a human adipocyte cell).

13. The construct according to claim 1, wherein the construct comprises (or is formed from) a population of cells, each cell type being included in a proportion that facilitates and/or promotes the growth and/or development of a construct that mimics the in vivo cancer microenvironment.

14. The construct according to claim 1, wherein the construct comprises or is formed from:
(i) cancer cells in an amount between about 20% and 90%, between about 30% and 80%, between about 40% and 70%, between about 45% and 65%, or between about 50% and 60% of the cell population;
(ii) cancer stem cells in an amount between about 0.5% and 5%, between about 1% and 2.5%, or about 2% of the cell population;
(iii) cancer associated fibroblast cells in an amount between about 2.5% and 15%, between about 5% and 10%, or between about 2.5% and 7.5% of the cell population;
(iv) a supportive cell in an amount between about 0% and 30%, between about 10% and 20%, or about 15% of the cell population; and/or
(v) a specialized cell to support a specific microenvironment in an amount between about 10% and 50%, between about 20% and 40%, between about 25% and 30%, or about 28% or about 23% of the cell population.

15. The construct according to claim 1, wherein the construct comprises or is formed from a population of cells comprising:
(a) about 60% cancer cells (e.g. glioblastoma cells);
(b) about 2% cancer stem cells (e.g. glioblastoma cancer stem cells);
(c) about 10% cancer associated fibroblast cells (e.g. glioblastoma cancer associated fibroblast cells); and
(e) about 28% of a specialized cell type to provide a specific microenvironment (e.g. microglia).

16. The construct according to claim 1, wherein the construct comprises or is formed from a population of cells comprising:
(a) about 50% cancer cells (e.g. glioblastoma cells);
(b) about 2% cancer stem cells (e.g. glioblastoma cancer stem cells);
(c) about 5% cancer associated fibroblast cells (e.g. glioblastoma cancer associated fibroblast cells);
(d) about 15% supportive cells (e.g. astrocytes); and
(e) about 28% of a specialized cell type to provide a specific microenvironment (e.g. microglia).

17. The construct according to claim 1, wherein the construct comprises or is formed from a population of cells comprising:
(a) about 70% cancer cells (e.g. breast cancer cells, such as triple negative breast cancer cells);
(b) about 2% cancer stem cells (e.g. breast cancer stem cells);
(c) about 5% cancer associated fibroblast cells (e.g. breast cancer associated fibroblast cells); and
(d) about 23% of a specialized cell type (e.g. adipocyte cells, such as human adipocyte cells).

18. The construct according to claim 3, wherein the extracellular matrix comprises one or more of: an alginate-based material (e.g. sodium alginate); cellulose (e.g. nanofibrillar cellulose); and extracellular proteins (e.g. laminin).

19. A method of making a construct for mimicking an in vivo environment, such as an in vivo cancer microenvironment, wherein the method comprises depositing on a surface:
(a) a cancer cell;
(b) a cancer stem cell;
(c) a cancer associated fibroblast cell; and optionally,
(f) a specialized cell type to provide a specific microenvironment.

20. The method of claim 19, further comprising depositing one or more of the following:
(e) a supportive cell;
(g) an immune cell; and
(h) an endothelial cell.

21. The method of claim 19, wherein cells (a) to (c) (with optionally one or more of cells (e) to (h)) are deposited on the surface together with:
(d) an extracellular matrix.

22. The method of claim 19, wherein the step of depositing comprises printing (e.g. bioprinting) the cells, and optionally the extracellular matrix, on to the surface.

23. The method of claim 19, comprising printing a plurality of constructs at a series of defined and/or discrete locations on a surface, optionally in a predetermined pattern to provide an array or microarray of constructs.

24. The method of claim 19, comprising:
(i) depositing or printing spheroids on to the surface;
(ii) depositing or printing cells on to the surface and then subsequently culturing the cells to form spheroids within the deposited cells or printed cells; or
(iii) depositing or printing a mixture of spheroids and single cells on to the surface.

25. The method of claim 24, further comprising magnetic bioprinting, optionally wherein the magnetic bioprinting is used to print cells into spheroids before or after a deposition or printing step.

26. The method of claim 19 further comprising culturing the construct for a period of time such that the construct mimics an in vivo cancer microenvironment

27. The method of claim 19, comprising printing droplets of a bioink formulation on to the surface, wherein the bioink formulation is made up of the cells (a) to (c) and optionally one or more of cells (e) to (h)) that have been pre-mixed with and/or are suspended in the extracellular matrix (d).

28. The method of claim 27, wherein between about 45% and 75% of the total volume of the bioink formulation is comprised of the suspension of cells and/or spheroids and/or between about 55% and 25% of the total volume of the bioink formulation is comprised of the extracellular matrix.

29. A bioink formulation for use in making a construct according to claim 1, comprising at least one of each of the following:
(a) a cancer cell;
(b) a cancer stem cell; and
(c) a cancer associated fibroblast cell; and optionally
further comprising one or more of the following:
(e) a supportive cell;
(f) a specialized cell type to provide a specific microenvironment;
(g) an immune cell; and/or
(h) an endothelial cell; and further optionally
comprising an extracellular matrix.

30. A method for testing the effects of a test agent or drug on a cell, for example a cancer stem cell, said method comprising:
providing a construct of claim 1;
maintaining a cell (e.g. CSC) within said construct;
contacting the cell with a test agent; and
determining the response of the cell (e.g. CSC) to the test agent.

31. A kit comprising at least one of each of the following:
(a) a cancer cell;
(b) a cancer stem cell; and
(c) a cancer associated fibroblast cell; and
further comprising one or more of:
a culture medium;
instructions for use; and
a substrate defining a deposition or print surface

32. The kit of claim 31, further comprising one or more of the following:
(d) an extracellular matrix;
(e) a supportive cell;
(f) a specialized cell type to provide a specific microenvironment;
(g) an immune cell; and/or
(h) an endothelial cell.

33. The kit of claim 31, wherein the components (a) to (c) and optionally one or more of components (d) to (h) are:
supplied and/or stored separately within the kit; or
are provided in the kit as a bioink formulation.
